(19) United States
(12) Patent Application Publication
(10) Pub. No.: US 2005/0256510 A1
(43) Pub. Date: Nov. 17, 2005

(54) Ventriculo-sinus shunting for disease treatment

(75) Inventors: Ari Moskowitz, Palo Alto, CA (US); William J. Bertrand, Ventura, CA (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/833,810

(22) Filed: Apr. 28, 2004

(51) Int. Cl. A61M 5/00; A61M 1/00; A61M 27/00

(52) U.S. Cl. 604/540; 604/541

(57) ABSTRACT

A method for treating a disease associated with increased concentration of an undesirable and/or deleterious agent in a central nervous system (CNS) is disclosed. A catheter having no flow restrictor member is placed is used to shunt cerebrospinal fluid (CSF) from a patient's cerebral ventricle to a venous sinus within the patient's head. Physiologically based pressure differential and control mechanisms between the ventricle and the venous sinus are exploited to control ventriculo-sinus flow of CSF.

### FIELD

The disclosure relates to shunting of cerebrospinal fluid (CSF) fluid and, more particularly, to shunting of CSF to a sagittal sinus for treating a disease associated with increased concentration of an agent in CSF.

### BACKGROUND

Increased concentrations of certain undesirable or deleterious agents in the central nervous system (CNS) of patients have been associated with disease states. For example, elevated levels of beta A4-amyloid, beta-2 microglubulin, and tau have been found in CSF of patients with Alzheimer's type adult-onset dementia. It has been proposed that removal of such agents from the CNS, particularly the CSF, may be beneficial for treating CNS diseases. For example, U.S. Pat. No. 5,334,315 teaches that a bodily fluid, such as CSF, may be removed from a patient, treated to remove an undesirable or deleterious substance, and returned to the patient to treat, e.g., Guillain-Barré syndrome. U.S. Pat. Nos. 5,980,480 and 6,264,625 teach that adult-onset dementia of the Alzheimer's type may be treated by removing a portion of a patient's CSF. See, e.g., the respective abstracts. U.S. Pat. Nos. 5,980,480 and 6,264,625 also teach an apparatus for removing CSF including (1) a conduit with a first opening and a second opening, the first opening of the conduit being adapted to be disposed in fluid communication with a space within a patient's arachnoid membrane, the second opening being adapted to be disposed in fluid communication with another portion of the patient's body; and (2) a flow rate control device attached to the conduit. See, e.g., the respective abstracts.

However, the prior teachings associated with removal of CSF for treating a disease associated with increased CSF concentrations of deleterious or undesirable agents teach removal with a device whose components are prone to malfunction, subject to wear and tear, and/or require difficult judgment on part of the physician who implants the device as to determine the proper flow control rate. For example, U.S. Pat. No. 6,264,625 teaches an apparatus having a flow rate control device. The flow rate control device may include, e.g., a clamp, pump, or valve. Use of a clamp to control flow rate may leave a physician guessing as to the appropriate size clamp to use, use of a pump may result in unnecessary and increased expense and to failure due to wear and tear, and use of a valve to control flow rate is similarly subject to wear and tear and failure over prolonged use.

Shunt systems and methods of shunt placement that do not require flow rate control devices have been described. For example, El-Shafei and El-Shafei have described the use of a valveless shunting catheter for treatment of hydrocephalus. Child's Nerv. Syst. (2001) 17:457-465. In this article, El-Shafei and El-Shafei teach that a method of placing one end of a shunt catheter into the ventricle of a patient and placing the other end of the catheter into the superior sagittal sinus (SSS) of the patient in a direction retrograde to blood flow results in a system that utilizes the impact pressure of the bloodstream in the SSS to maintain an intraventricular pressure more than the sinus pressure, regardless of posture or intrathoracic pressure. However, El-Shafei and El-Shafei do not teach that a retrograde ventriculosinus shunt would be beneficial to treating a disease associated with an increased concentration of a deleterious or undesirable agent in the CSF.

### BRIEF SUMMARY OF THE INVENTION

The present invention in various embodiments advantageously utilizes the body's natural control processes to remove CSF for treating a disease associated with increased CSF concentrations of a deleterious and/or undesirable agent.

In an embodiment, the invention provides a method for treating a patient at risk of or suffering from a disease associated with increased concentration of an agent in the patient's CNS. The method comprises selecting a patient suffering from or as risk of the disease and draining the patient's CSF from a cerebral ventricle of the patient to a venous sinus in the patient's head.

An embodiment of the invention provides a method for treating a patient at risk of or suffering from a disease associated with increased concentration of an agent in the patient's CNS. The method comprises selecting a patient suffering from or as risk of the disease.

The method further comprises inserting a first end of a drainage catheter into a cerebral ventricle of the patient and inserting a second end of the drainage catheter into a venous sinus of the patient head, to allow the patient's CSF to flow through the catheter from the ventricle to the venous sinus. The second end of the drainage catheter may be inserted into the venous sinus in a retrograde direction facing upstream of blood flow in the venous sinus.

The present invention provides several advantages over previously described methods and apparatus to remove CSF for treating a disease associated with increased concentrations of a deleterious and/or undesired agent from the CNS. For example, use of a shunting system having no flow restrictor is less subject to wear and tear than shunts having flow restrictors and is likely to perform desirably for extended periods of time. In addition, control of CSF flow through a shunting catheter that uses the body's own control mechanisms provides physiological-based flow control rather than mechanical-based flow control. For example, drainage of CSF to, e.g., the sagittal sinuses occurs naturally and is driven by a pressure differential between intrasinus pressure and intraventricular pressure that is generally maintained regardless of posture, etc. Rather than attempting to approximate such physiologically based mechanisms to control CSF flow, such as with shunts having flow restrictors, the present invention in various embodiments uses the body's own control mechanisms to shunt fluid from a ventricle into a venous sinus. In addition, using the naturally occurring pressure difference between a venous sinus and a cerebral ventricle allows for flow rates to change as the patient's physiological control mechanisms dictate, which is advantageous over shunts with flow restrictors that can be relatively inflexible in the amount of CSF that may flow. Increased reliability and physiologically based control are but a few advantages the present invention offers with regard to removal CSF for treating a disease associated with increased concentrations of a deleterious and/or undesired agent from the CNS. These and other advantages will be apparent to one of skill in the art upon reading the disclosure presented herein.

### BRIEF DESCRIPTION OF THE DRAWINGS

FIG. 1 is a flow of a method of an embodiment of the present invention;

FIG. 2 is an illustration of a shunt system in accordance with certain embodiments of the present invention;

FIG. 3 is a flow of a method of an embodiment of the present invention;

FIG. 4 is an illustration of a shunt system in accordance with certain embodiments of the present invention installed in the cranium of a patient;

FIG. 5 is a side view of a ventricular catheter used in the shunt system illustrated in FIG. 4;

FIG. 6A is a top view of a valve used in the shunt system illustrated in FIG. 4;

FIG. 6B is a side view of a valve used in the shunt system illustrated in FIG. 4;

FIG. 7 is a side view of a sinus catheter used in the shunt system illustrated in FIG. 4;

FIG. 8 is a side view of a right angle clip used in the shunt system illustrated in FIG. 4;

FIG. 9 is a cross-sectional view a ventricular catheter inserted into a dural hole formed in accordance with embodiments of the present invention;

FIG. 10 is a side view of a ventricular catheter stretcher used in installation used of the shunt system illustrated in FIG. 4;

FIG. 11 is a side view of a female luer used in an embodiment of a ventricular catheter used in the shunt system illustrated in FIG. 4;

FIG. 12 is a side view of a clamp used in installation of the shunt system illustrated in FIG. 4;

FIG. 13 is a flow of a method of an embodiment of the present invention;

FIG. 14 is a flow of a method of an embodiment of the present invention; and

FIG. 15 is a flow of a method of an embodiment of the present invention.

The drawings are not necessarily to scale.

### DETAILED DESCRIPTION OF THE INVENTION

In the following descriptions, reference is made to the accompanying drawings that form a part hereof, and in which are shown by way of illustration of several specific embodiments of the invention. It is to be understood that other embodiments of the present invention are contemplated and may be made without departing from the scope or spirit of the present invention. The following detailed description, therefore, is not to be taken in a limiting sense.

While not intending to be bound by any particular theory, the present invention in various embodiments is based, in part, on a premise that devices and methods that use of the body's natural control processes to remove CSF from a cerebral ventricle for treating a disease associated with increased CNS concentrations of a deleterious and/or undesirable agent are advantageous over devices and methods that provide active mechanisms to remove CSF or use flow restrictors to control the rate at which CSF may be removed.

In addition and not intending to be bound by any particular theory, various embodiments of the invention are based, in part, on the premise that devices and methods that use of the body's natural control processes to remove CSF from a cerebral ventricle for treating a disease associated with increased CNS concentrations of a deleterious and/or undesirable agent are advantageous over devices and methods that do not take advantage of the body's natural control processes.

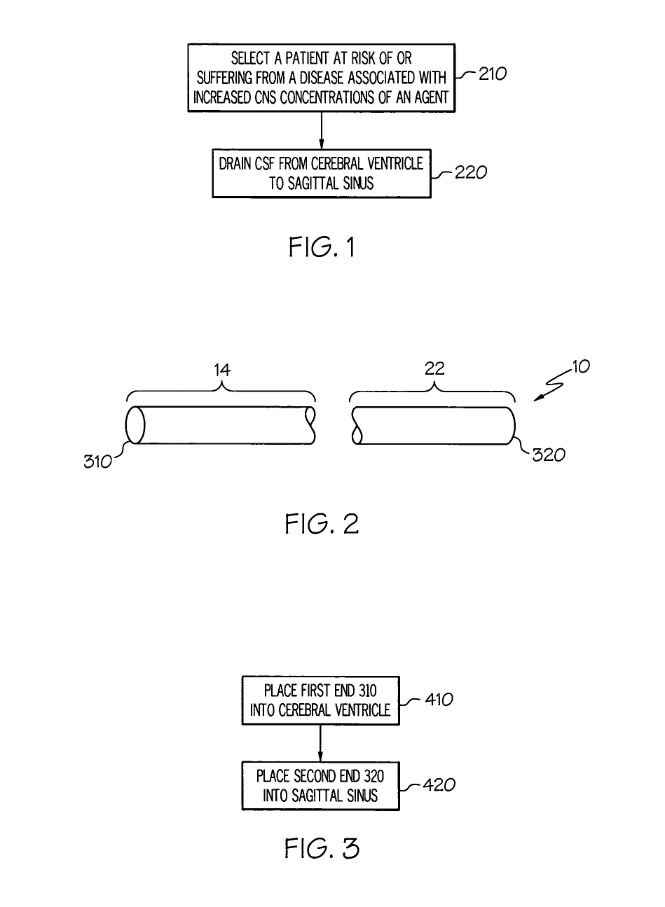

A method of an embodiment of the invention is illustrated in FIG. 1. As shown at 210, the method comprises selecting a patient at risk of or suffering from a disease associated with an increased concentration of a deleterious and/or undesirable agent in the patient's CNS. The increased concentration of the agent may be in a population of patients having the disease relative to a population of people not having the disease. Alternatively, the increased concentration of the agent may be in the selected patient relative to a population of people not having the disease. The increased CNS concentration may be an increased concentration in CSF. As shown at 220, the method further comprises shunting CSF from a cerebral ventricle of the patient to a sagittal sinus of the patient. The ventricle may be a lateral ventricle. The saggital sinus may be a superior sagittal sinus.

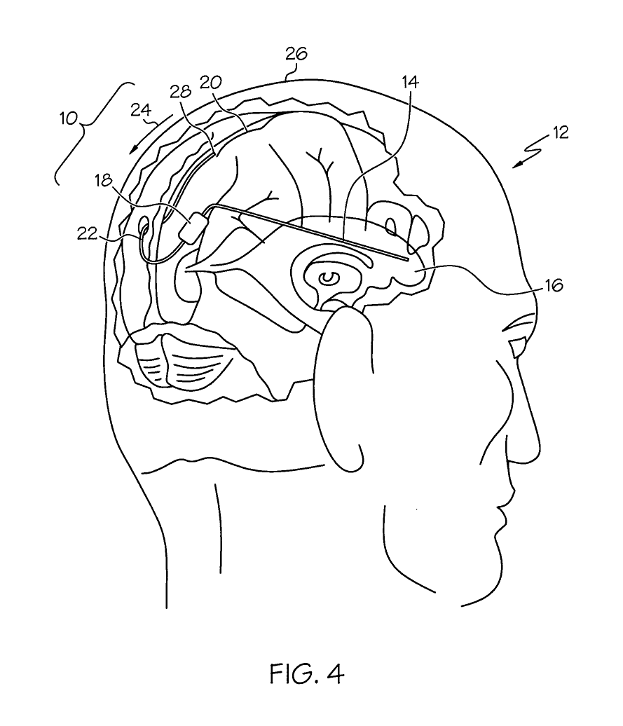

Referring to FIG. 2, various methods of the invention may be performed by using a shunting system 10 as depicted. The shunting system comprises a catheter, which comprises a ventricular portion 14 and a sinus portion 22. The ventricular portion comprises a first end portion 310 and the sinus potion 22 comprises a second end portion 320. As shown FIG. 3 and according to an embodiment of the invention, the draining 220 may be accomplished by inserting the first end portion 310 into a cerebral ventricle of a patient and inserting the second end portion 320 into a sagittal sinus of the patient to allow the patient's CSF to flow through the catheter from the ventricle to the sagittal sinus. Because of the close proximity of the sagittal sinuses to the cerebral ventricles and thus relatively small pressure differences to gravitation and because of naturally occurring reabsorption of CSF into the sagittal sinuses and thus physiologically controlled mechanisms, the shunting catheter 300 need not include a flow restrictor element. To further enhance the performance of the shunt system, the second end portion 320 of the catheter 300 may be inserted into the sagittal sinus in a retrograde direction facing upstream of blood flow in the sagittal sinus. Such placement reduces the likelihood of clotting and utilizes the impact pressure of the bloodstream in the sagittal sinus to maintain an intraventricular pressure more than the sinus pressure. For patient safety, it may be desirable that the shunting system comprise a unidirectional check valve to allow flow of CSF through the catheter from the ventricle to the sinus and to prevent flow of blood from the sinus to the ventricle.

Any disease associated with an increased CNS concentration of a deleterious and/or undersirable agent may be treated according to various embodiments of the invention. In the context of the present invention, the terms “treat”, “therapy”, and the like are meant to include methods to alleviate, slow the progression, prevent, attenuate, or cure the treated disease. Non-limiting examples of diseases associated with increased CNS concentrations of a deleterious and/or undesirable agent that may be treated according to various embodiments of the invention include adult-onset dementia of the Alzheimer's type, Guillain-Barré syndrome; Multiple Sclerosis (MS); Amyotrophic Lateral Sclerosis (ALS); Acquired Immune Deficiency Syndrome (AIDS); demential complex; encephalopathy, such as from rabies and bovine spongiform encephalopathy; encephalitis; meningitis; polio; tetanus; CNS infection; and autoimmune disease.

Any deleterious and/or undesirable agent may be removed from the CNS by removing CSF according to various embodiments of the invention. In the context of the present invention, an undesirable and/or deleterious agent is an agent whose presence in the CNS is associated with a disease or an agent whose increased presence in the CNS is associated with a disease. Deleterious and/or undesirable agents that may be removed include, but are not limited to, proteins, polypeptides, interleukins, immunoglobulins, proteases, interferon, tumor necrosis factor, complement, complement associated factors, gliotoxic factors, leucocytes, lymphocytes, prions, viruses, and single celled organisms, such as fungi, bacteria, and protozoa. Examples of proteins that may be removed include A4-amyloid, beta-2 microglubulin, and tau.

Any shunt system or catheter 300 may be used according to methods of various embodiments of the invention. Preferably, the shunt system 300 does not include a flow restrictor element.

It will be understood that a lubricious material may be disposed on or about at least a portion of a component of a shunt system 10. Disposing a lubricious material on or about, e.g., a ventricular portion 14 and/or a sinus portion 22 of a drainage catheter may facilitate insertion of the catheter into the ventricle and/or the sinus. The lubricious material may be disposed on or about an exterior surface or the lumen of the drainage catheter. Any known or future developed lubricious material, or combinations thereof, may be used. Preferably, the lubricious materials are medically suitable for inserting into a patient. Examples of suitable lubricous materials that may be disposed on at least a portion of a component of a shunt system 10 include fluoroethylpolymer, polytetrafluoroethylene (PTFE), polyetheretherketone (PEEK), ethylene tetrafluoroethylene (ETFE), paralene, a hydrophilic polymer, and the like. Additional examples of suitable coating that may be applied include those described in the following patents and patent publications: US 20040030159; U.S. Pat. No. 6,558,734, U.S. Pat. No. 6,278,018; U.S. Pat. No. 6,603,040; U.S. Pat. No. 6,669,994; WO0121326; WO 0144174; and WO 2003055611. In an embodiment, the lubricious material is a hydrogel. The hydrogel may be a polyvinyl pyrrolidone (PVP) hydrogel, such as Medtronic's BIOGLIDE. In addition to facilitating insertion of a catheter, a lubricious material such as a hydrogel may prevent infection, thrombosis and catheter occlusion. For example, BIOGLIDE technology has been shown to resist protein deposition, adherence of thrombosis, and reduce platelet and complement activation and may also inhibit tissue adherence.

To further prevent thrombosis, infection, and/or occlusion, an antimicrobial agent and/or an anticoagulant agent may be incorporated into or on the catheter material and/or the lubricious material. Any antimicrobial agent, such as an antibacterial agent, an antiseptic agent, etc., may be used to prevent infection. Non-limiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver sulfadiazine and alcohols. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include those listed above, as well as minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Any anticoagulant agent, such as heparin, streptokinase, and/or urokinase, may be used to prevent thrombosis. If an anticoagulant is incorporated into or on a drainage catheter, it is desirable that the anticoagulant be incorporated into or on a sinus portion 22 of the catheter.

An antimicrobial agent and/or an anticoagulant may be incorporated into or on catheter material or a lubricious material using any know or future developed technique. For example, the antimicrobial agent and/or anticoagulant agent may be disposed in or on the catheter or lubricious material through compounding or solvent expansion/swelling techniques. A hydrogel or a catheter comprising a hydrogel, for example, may be presoaked in a solvent comprising the agent to incorporate the agent. Alternatively, an antimicrobial agent or anticoagulant agent may be covalently attached to a catheter or coating material using any known or future developed technology. Suitable technology includes Surmodic's PHOTOLINK technology. Conventiaional TDMAC (Tridodecylmethylammonium) coating technology, such as with TDMAC-heparin (Tridodecylmethylammonium heparinate), may also be employed. Additional technology for incorporating a therapeutic agent into or on a catheter that may be used in accordance with the teachings of the present invention are discussed in, for example, U.S. Pat. No. 6,303,179, U.S. Pat. No. 6,143,354, US 2004/0039437, and WO 04/014448. Of course any other therapeutic agent may be incorporated into or on the catheter or lubricious coating.

The following description relates to exemplary catheters, shunt systems, and methods that may be employed according to the teachings of the invention.

FIG. 4 shows a ventricular to sagittal sinus shunt system 10 in place in a patient 12. Ventricular catheter 14 has been inserted through a burr hole (not shown) into the lateral ventricle 16 of patient 12. Ventricular catheter 14 is coupled to valve 18, such as a unidirectional check valve, which allows flow of CSF from lateral ventricle 16 to sagittal sinus 20, but prevents flow of blood from the sagittal sinus 20 to the lateral ventricle 16. Valve 18 is also coupled to sinus catheter 22 shown inserted through another burr hole (also not shown) into the superior sagittal sinus 20.

Shunt system 10 allows CSF present in lateral ventricle 16 to flow through ventricular catheter 14, valve 18 and sinus catheter 22 into the blood stream of sagittal sinus 20 where the excess CSF can be reabsorbed into the body. The vertical distance between the location of ventricular catheter 14 and sinus catheter 22 is small compared with vertical distance usually associated with a peritoneum catheter leading to smaller pressure differences due to gravitation between the inlet catheter, ventricular catheter 14, and the outlet catheter, sinus catheter 22.

Blood flow in sagittal sinus 20 is generally from in the direction shown by arrow 24 from the frontal portion of cranium 26 of patient 12 to the rear portion of cranium 26 of patient 12. In a preferred embodiment, distal end 28 sinus catheter 22 has a retrograde orientation in sagittal sinus 20, essentially pointing upstream against the flow of blood in sagittal sinus 20 shown by blood flow arrow 24. Positioned in this manner, outlet of CSF from distal end 28 of sinus catheter 22 provides a collision vortex in the flow of blood around sinus catheter 22. This retrograde position provides a substantial decrease in the likelihood of thrombosis resulting from an ante grade position of distal end 28 of sinus catheter 22 in the wake created by sinus catheter 22 of the bloodstream in sagittal sinus 20.

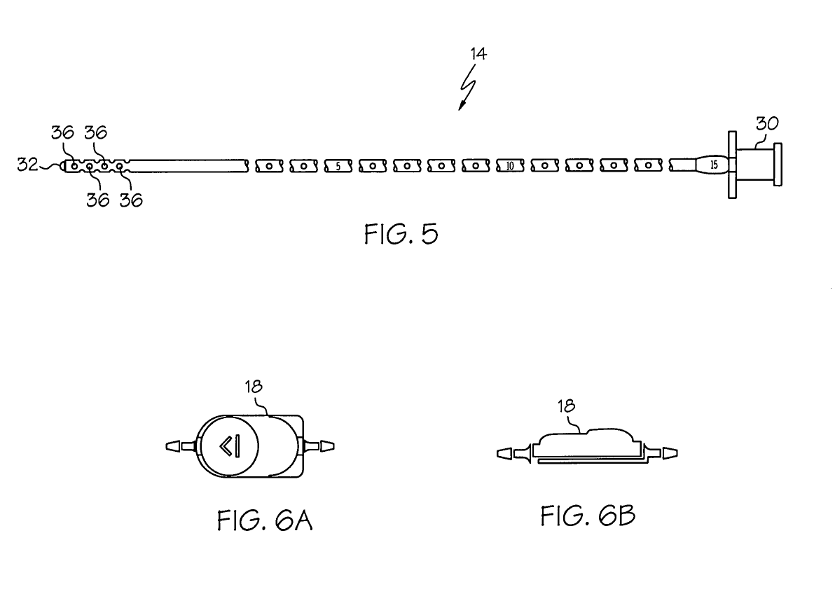

Ventricular catheter 14 is illustrated more clearly in FIG. 5 coupled with female luer 30 (also shown in FIG. 11). Ventricular catheter 14 is an extensible elongate body having distal end 32 and proximal end 34. Distal end 32 of ventricular catheter 14 is inserted into lateral ventricle 16 of cranium 26 of patient 12 as will be discussed below. Ventricular catheter 14, shown in a relaxed state, has an outside diameter of 2.5 millimeters and a length of 15 centimeters. Ventricular catheter 14 has a lumen with a diameter of 1.3 millimeters (relaxed state). Distal end 32 contains outlets 36 from the lumen consisting of four rows of four holes each extending approximately 1 centimeter from distal end 32. Ventricular catheter 14 has 13 length markers in one centimeter spacing from 3 centimeters to 15 centimeters from proximal end 34 including numerical length markers at 5, 10 and 15 centimeters. Such length markers aid the surgeon in determining how deeply ventricular catheter 14 is placed. Female luer 30 is sewn onto proximal end 34 of ventricular catheter 14. Ventricular catheter 14 is formed of an extensible material such as silicone elastomer tubing having a durometer of fifty (50) and an elongation of four hundred fifty percent (450%). Ventricular catheter 14 has a tensile strength of 900 pounds per square inch.

Valve 18 (FIG. 6A and FIG. 6B) is a one-way check valve approximately 20 millimeters long, 11 millimeters wide and 4 millimeters high. Valve 18 only ensure one way flow from ventricle 16 to sagittal sinus 20 and doesn't regulate the rate of flow of CSF. Valve 18 may have an opening pressure of, e.g., less than or equal to about 6 cm/H₂O, less than or equal to about 5 cm/H₂O, or less than or equal to about 4 cm/H₂O, under physiological flow production rates of approximately 20 ml/hr, e.g., 20.4 ml/hr.

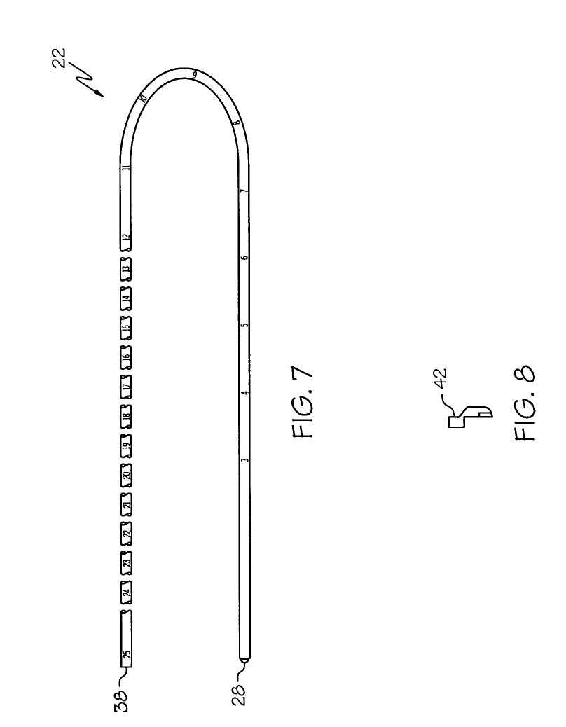

Sinus catheter 22 in FIG. 7 has distal end 28 having a smooth open-ended tip and proximal end 38. Sinus catheter 22 is formed of a semi-rigid material such as silicone elastomer tubing having a durometer of eighty (80) with an outside diameter of 2.1 millimeters and a length of 25 centimeters. Sinus catheter 22 has a lumen with a diameter of 1.2 millimeters. Sinus catheter 22 has 23 numeric length markers in one centimeter spacing from 3 centimeters to 25 centimeters from distal end 28.

In order to properly insert sinus catheter 22 in a retrograde position in sagittal sinus 20, sinus catheter 22 has bend 40 located approximately seven (7) centimeters from distal end 28. As is shown in FIG. 4, bend 40 allows sinus catheter 22 to lie smoothly against head of patient 12 once inserted into sagittal sinus 20. Bend 40 actually makes it difficult for the surgeon to insert sinus catheter 22 in a position other than retrograde essentially ensuring proper placement of sinus catheter 22 in sagittal sinus 20. While bend 40 is illustrated to be approximately a one-hundred eighty degree bend, other degrees of bend are possible and contemplated. Bend 40 alternatively could be a ninety degree bend and achieve similar results. It is preferred that bend 40 be at least a ninety degree bend.

Shunt system 10 is installed by first drilling a burr hole in cranium 26 using a conventional technique. In some patients, such as small children and/or infants, a burr hole may not be necessary. A parieto occipital skin flap is mapped to expose the sites of sinus exposure and the dural hole for ventricular catheter 14 insertion into lateral ventricle 16. The sinus will be exposed anterior to the external occipital protuberance and the opening to penetrate the ventricle 16 will be made lateral and slightly anterior to the exposed sinus, in line with the lateral ventricle 16. Two separate curvilinear small incisions may be used instead of the skin flap in patients above six years of age, to access the superior sagittal sinus 20 and lateral ventricle 16, respectively. Alternatively, a frontal approach to access lateral ventricle 16 could be used.

After reflection of the scalp, the tissue is incised over the sites chosen for the bony openings exposing the superior sagittal sinus 20 and cerebral ventricles, respectively.

The superior sagittal sinus 20 is exposed through a burr hole centered over the sagittal suture. The burr hole may be widened to expose the sinus fully, which in some instances may deviate slightly to the right of the sagittal suture, and bevel its posterior edge.

A burr hole may be made in line with the lateral ventricle 16, exposing a circle of dura mater. If right angle clip 42 (FIG. 8) is not used, it is recommended that the posterior rim of the burr hole be beveled where catheter 14 emerges and curves to lie adjacent to the skull. A subgaleal pocket should be formed with appropriate depth to accept the extracranial portion of the ventricular catheter 14 and valve 18.

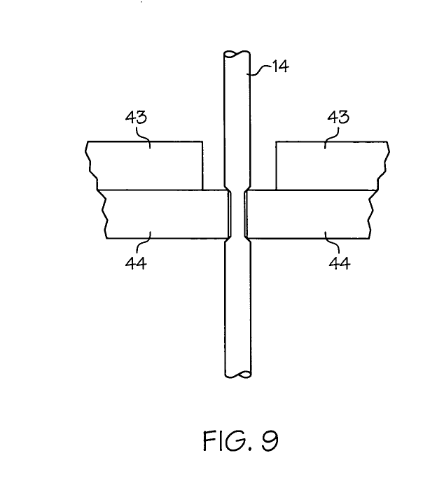

A burr hole will be made in skull 43 at the point of insertion of ventricular catheter 14. A hole also will be made in the dura having predetermined diameter as illustrated in FIG. 9. In order to help control CSF leakage cranium 26, ventricular catheter 14 is stretched from its relaxed state prior to insertion through dura 44. A hole with a precise diameter is made in dura 44 which, preferably, is approximately the diameter of ventricular catheter 14 in its relaxed state. In order to be able to insert ventricular catheter 14 through dura 44, ventricular catheter 14 is stretched in a controlled manner in order to reduce its outside diameter to a diameter which is less than the controlled diameter of the hole made in dura 44. Ventricular catheter 14 is inserted through dura 44 in its stretched state allowing easy insertion. Following insertion, ventricular catheter 14 reverts to its relaxed state allowing its outside diameter to return to approximately equal to or smaller than its original relaxed state diameter and essentially filling the hole in dura 44. Having a controlled shape and diameter for the hole created in dura 44 allows ventricular catheter, once inserted, to mostly fill and seal the hole in dura 44 helping to prevent or control leakage of CSF from inside cranium 26.

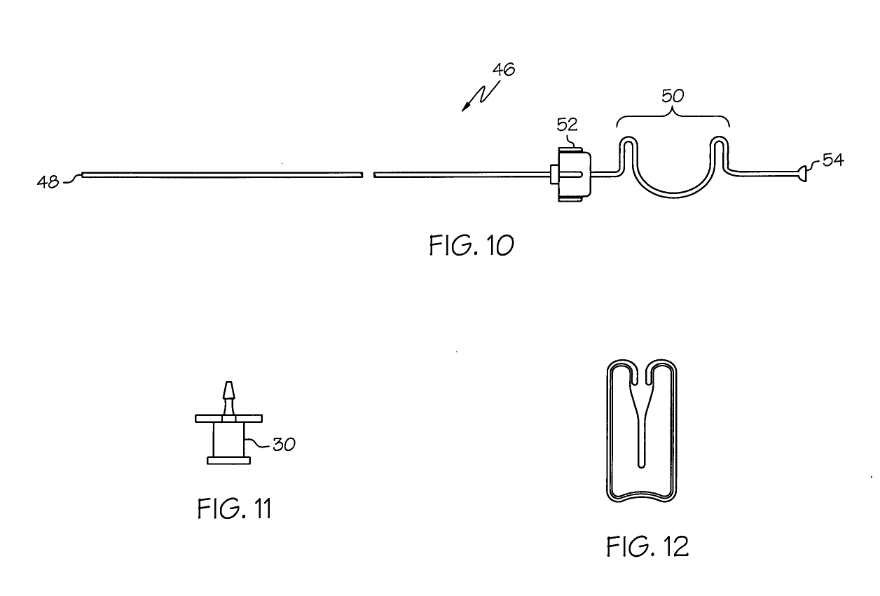

Catheter stretcher 46 (FIG. 10) can be utilized to controllably stretch ventricular catheter 14 to a stretched state in which the outside diameter of ventricular catheter has been made smaller to allow ventricular catheter 14 to be easily inserted through a controlled diameter hole in dura 44. Catheter stretcher 46 consists of an elongate rod having a diameter smaller than the diameter (1.3 millimeters) of the lumen of ventricular catheter 14 allowing distal end 48 to be inserted through female luer 30 into lumen of ventricular catheter 14. Distal end 48 of catheter stretcher 46 penetrates the lumen of ventricular catheter completely with distal end 48 of catheter stretcher resting against distal end 32 of ventricular catheter 14. Curves 50 in catheter stretcher 46 make catheter stretcher 46 easier to handle. Luer cap 52 is affixed on catheter stretcher 46 a distance away from distal end 48 which is greater than the distance between distal end 32 of ventricular catheter 14 and female luer 30. Once catheter stretcher 46 is inserted completely into lumen of ventricular catheter 14, female luer 30 is grasped and pulled up and mated with luer cap 52. The amount that distance between distal end 48 and luer cap 52 exceeds the distance between distal end 32 and female luer 30 is the controlled amount which ventricular catheter 14 is stretched. As ventricular catheter 14 is stretched its outside diameter becomes smaller.

Catheter stretcher 46 also provides ventricular catheter 14 with stiffness to aid in insertion of ventricular catheter 14 into lateral ventricle 16.

A small hole with a diameter greater than outer diameter of ventricular catheter 14 in its stretched state and less than outer diameter of ventricular catheter 14 in its relaxed state is made in the center of exposed dura mater 44.

Catheter stretcher 46 has, at its proximal end, tip 54 which is sized and shaped at a desired diameter for the dural hole. Preferably, this diameter is greater than outer diameter of ventricular catheter 14 in its stretched state and less than outer diameter of ventricular catheter 14 in its relaxed state. Preferably, tip 54 is hemispherically shaped.

Once the dura has been exposed, tip 54 can be applied against the dura and a diathermy current applied to catheter stretcher 46, typically by touching a cautery needle to the shank of catheter stretcher 46 near tip 54 in order to cauterize dura 44 creating a hole in dura 44 of the precise size and shape of tip 54. Since tip 54 is sized and shaped to the desired size and shape of the dural hole, tip 54 need not be manipulated to manually create a hole of a size larger than a cautery tip typically used. Such undesirable manual manipulations tend to create irregular and unevenly sized holes which vary from surgery to surgery.

Right angle clip 42 on ventricular catheter 14 can be used as a marker for planned depth of ventricular catheter 14 insertion by sliding it the desired distance from proximal end 34 of ventricular catheter 14 prior to insertion.

Stretched ventricular catheter 14 is introduced into the lateral ventricle 16 through the dural opening (the direction of ventricular catheter insertion is along a line extending from the dural hole to the ipsilateral pupil) into the anterior horn. The position of the catheter stretcher (stylet) is maintained with one hand and luer cap 52 is unlocked with the other hand allowing ventricular catheter to relax to its original diameter without retracting from ventricle 16. Ventricular catheter 14 should fit snugly in the dural hole, helping to hermetically seal it. Imaging may be used to verify proper placement of the catheter.

The stylet (catheter stretcher) 46 is removed and ventricular catheter is clamped (with clamp 58 shown in FIG. 12) immediately after the withdrawal of stylet 46 to help prevent CSF loss.

Right angle clip 42 on ventricular catheter 14 may be used to bend ventricular catheter 14 to an approximate ninety degree angle where it exits the twist drill or burr hole. The extracranial portion of ventricular catheter is pressed into the split tubular segment of right angle clip 42 to form a right angle bend. Stretching of ventricular catheter 14 is avoided when it is pressed into right angle clip 42. It is recommended that right angle clip 42 be secured to adjacent tissue by passing sutures through the two suture flanges on the sides of right angle clip 42.

A clamp is removed as necessary and saline is injected into ventricle 16 through ventricular catheter 14 to replace lost CSF and to clear any tissue debris, to raise the CSF pressure and to help make sure that there is not leakage from around ventricular catheter 14.

The extra length of ventricular catheter 14 is cut off so that only two to three centimeters of ventricular catheter 14 remain projecting outward from the burr hole.

The inlet port of valve 18 is fit into the open end of ventricular catheter 14 and is secured by a suture.

The clamp is momentarily removed on ventricular catheter 14 to prime valve 18 and to remove air bubbles. The clamp is reapplied to ventricular catheter 14.

After exposing the roof of the sinus by direct observation or needle puncture, an opening is made through the dural roof of the sinus 20 large enough to accommodate sagittal sinus catheter 22. A finger is applied on the sinus 20 at the puncture site to prevent excessive blood loss.

Distal end 28 of sinus catheter 22 is introduced into sagittal sinus 20 and advanced forward against the direction of blood flow for a distance of approximately five centimeters. If any obstacle to the free passage of sinus catheter 22 is encountered, the sinus catheter 22 is withdrawn a bit and redirected in its advance into sagittal sinus 20. Sinus catheter 22 is advanced slightly and retracted to approximately five centimeters to provide additional assurance that sinus catheter 22 resides in the main sagittal sinus 20 lumen.

After placement of sinus catheter 22, good blood flow is checked by allowing venous back flow into the unclamped sinus catheter 22. After establishing venous back flow, saline is injected into sinus catheter 22 to clear blood from sinus catheter 22. The sinus catheter 22 is clamped. Any bleeding from around sinus catheter 22 should be controlled, e.g., by gel foam, pressure and/or suture.

The proximal end 38 of sinus catheter 22 is formed in a smooth U-curve to the outlet of valve 18. The required length of proximal end 38 of sinus catheter 22 is estimated, the position of the clamp on sinus catheter 22 is adjusted and the extra sinus catheter 22 is cut off.

The outlet port of valve 18 is fit into proximal end (having been cut off) of sinus catheter 22 and secured by a suture. Valve 18 is secured by suture to the underlying pericranium.

The clamps on ventricular catheter 14 and sinus catheter are removed, respectively, allowing CSF to flow in shunt system 10. The skin is closed in the usual manner.

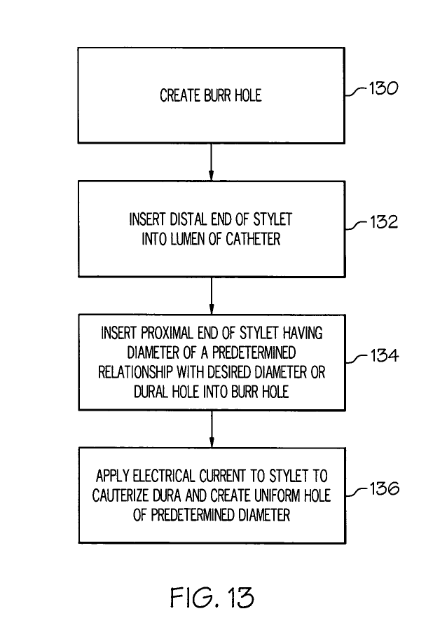

A method of an embodiment of the present invention is illustrated in FIG. 13. A burr hole of a predetermined diameter is made (130) in the dura 44 of patient 12. An extensible ventricular catheter 14 is stretched (132) to a predetermined distance to narrow its outside diameter. The stretched catheter 14 is inserted (134) through a hole smaller than a hole through which an unstretched ventricular catheter 14 would have easily fit. The ventricular catheter 14 is allowed to return (136) to its relaxed state.

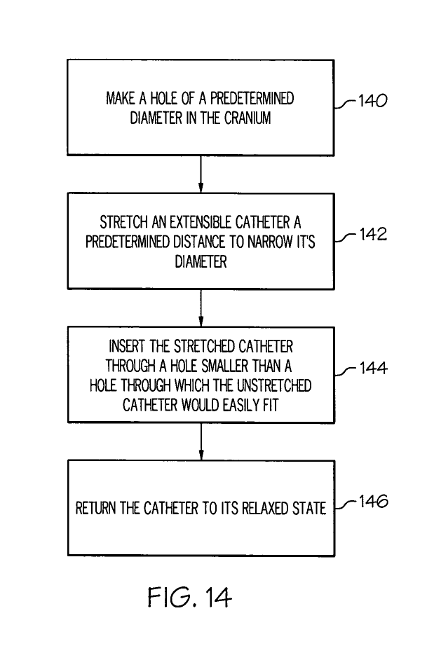

A method of an embodiment of the present invention is illustrated in FIG. 14. A burr hole in the skull is created (140). The distal end 48 of catheter stretcher (stylet) 46 is inserted (142) into the lumen of ventricular catheter. Proximal tip 54 of stylet 46 having a diameter having a predetermined relationship with a desired diameter of a hole being created in the dura is inserted (144) into the burr hole adjacent the dura. Electrical current is applied (146) to stylet to cauterize dura 44 and create a uniformly sized and shaped dural hole of a predetermined diameter.

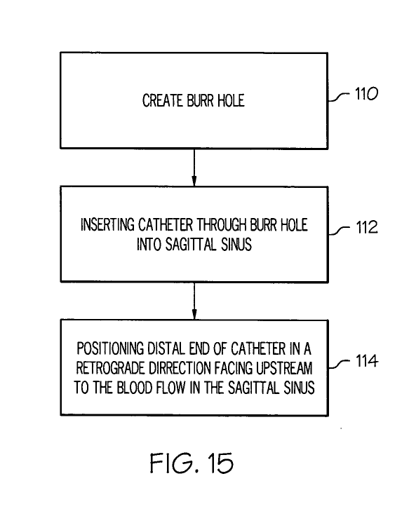

A method of an embodiment of the present invention is illustrated in FIG. 15. A burr hole is created (110) in the cranium 26 of the patient 12, if necessary. A catheter, such as sinus catheter 22, is inserted (112) through the dura into the sagittal sinus 20. The distal end of the catheter 22 is positioned in a retrograde direction facing upstream to the blood flow in the sagittal sinus 20.

Any combinations of the methods presented in the present disclosure may be used alone or in combination. For example, the methods presented in FIGS. 13-15 may be practiced sequentially according to an embodiment of the invention.

The following patent applications provide additional information regarding methods and apparatuses for placement of a ventriculosinus shunting catheter that may be used according to the teachings of the present invention: U.S. patent application Ser. No. 10/698,952 to Moskowitz for “Apparatus and method for cauterizing the dura of a patient using a dural patch”, filed Oct. 31, 2003; U.S. patent application Ser. No. 10/699,611 to Stepkowski et al. for “Stylet, apparatus and method for inserting a catheter into the dura of a patient by stretching the catheter”, filed Oct. 31, 2003; U.S. patent application Ser. No. 10/699,586 to Moskowitz et al. for “Apparatus and method for making a hole in the dura”, filed Oct. 31, 2003; and U.S. patent application Ser. No. 10/698,334 to Moskowitz et al. for “Apparatus and method for retrograde placement of sagittal sinus drainage catheter”, filed Oct. 31, 2003.

All scientific and technical terms used in this application have meanings commonly used in the art unless otherwise specified. The definitions provided herein are to facilitate understanding of certain terms used frequently herein and are not meant to limit the scope of the present disclosure.

All patents and technical papers cited herein are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

The preceding specific embodiments are illustrative of the practice of the invention. It is to be understood, therefore, that other expedients known to those skilled in the art or disclosed herein, may be employed without departing from the invention or the scope of the appended claims. For example, the present invention is not limited to the apparatus described herein per se, but other medical devices, such as shunting catheters, etc., may be employed to carryout the methods described herein. In addition, it will be understood that CSF may be drained to a venous sinus in the patient's head, other than a sagittal sinus, according to the teaching of the invention. Other suitable venous sinuses located within the head include the transverse sinus, Straight, inferior sagittal sinus, and cavernous sinuses

Thus, embodiments of the apparatus and method for removing CSF to treat a disease associated with a deleterious and/or undesirable agent in the CNS are disclosed. One skilled in the art will appreciate that the present invention can be practiced with embodiments other than those disclosed. The disclosed embodiments are presented for purposes of illustration and not limitation, and the present invention is limited only by the claims that follow.

1. A method for treating a disease associated with increased concentration of an agent in cerebral spinal fluid (CSF), using a drainage catheter having first and second end portions, the method comprising:
selecting a patient suffering from or as risk of the disease;
placing the first end portion of the catheter into a cerebral ventricle of the patient; and
placing the second end portion of the catheter into a venous sinus of the patient's head to allow the patient's CSF to flow through the catheter from the ventricle to the venous sinus.

2. The method of claim 1, wherein the venous is a sagittal sinus.

3. The method of claim 2, wherein the sagittal sinus is the superior sagittal sinus.

4. The method of claim 1, wherein the venous sinus is a transverse sinus.

5. The method of claim 1, wherein placing the second end portion of the catheter into the venous sinus of the patient comprises placing the second end portion of the catheter into the venous sinus in a retrograde direction facing upstream to blood flow in the venous sinus.

6. The method of claim 5, further comprising creating a hole in the patient's dura through which the catheter may be inserted to place the first end portion of the catheter in the cerebral ventricle.

7. The method of claim 6, further comprising stretching the catheter such that a stretched outer diameter of the catheter is less than the diameter of the hole in the dura to facilitate insertion of the catheter the hole.

8. The method of claim 7, further comprising relaxing the catheter after insertion through the hole in the dura and allowing the outer diameter of the catheter to expand from the stretched outer diameter to a relaxed outer diameter and to sealingly engage the hole.

9. The method of claim 8, wherein placing the first end portion of the catheter and placing the second end portion of the catheter comprise placing a catheter having no flow restrictor element.

10. The method of claim 8, further comprising operably connecting a sinus piece of the catheter to a ventricular piece of the catheter,
wherein the catheter comprises two pieces, the ventricular piece comprising the first end portion and the sinus piece comprising the second end portion.

11. The method of claim 10, further comprising operably connecting a unidirectional check valve to the sinus piece and to the ventricular piece, the unidirectional check valve adapted to allow the CSF to flow from the ventricle to the venous sinus and to prevent flow of the patient's blood from the venous sinus to the ventricle.

12. The method of claim 11, wherein connecting the unidirectional check valve comprises connecting a check valve having an opening pressure of less than or equal to about 6 cm/H₂O at a flow rate of about 20 ml/hr.

13. The method of claim 12, wherein the flow of CSF from the ventricle to the venous sinus is not impeded by a flow restrictor element.

14. The method of claim 13, wherein the disease is selected from the group consisting of Guillain-Barré syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

15. The method of claim 14, wherein the venous is a sagittal sinus.

16. The method of claim 15, wherein the sagittal sinus is the superior sagittal sinus.

17. The method of claim 14, wherein the venous sinus is a transverse sinus.

18. The method of claim 13, wherein the disease is Alzheimer's type adult-onset dementia.

19. The method of claim 18, wherein the venous is a sagittal sinus.

20. The method of claim 19, wherein the sagittal sinus is the superior sagittal sinus.

21. The method of claim 18, wherein the venous sinus is a transverse sinus.

22. The method of claim 1, wherein the disease is selected from the group consisting of Guillain-Barre syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

23. The method of claim 1, wherein the disease is Alzheimer's type adult-onset dementia.

24. A method for treating a disease associated with increased concentration of an agent in cerebral spinal fluid (CSF), comprising
selecting a patient suffering from or as risk of the disease;
draining the patient's CSF from a cerebral ventricle of the patient to a venous sinus of the patient's head.

25. The method of claim 24, wherein the disease is selected from the group consisting of Guillain-Barre syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

26. The method of claim 24, wherein the disease is Alzheimer's type adult-onset dementia.

27. A method for treating a disease associated with increased concentration of an agent in a central nervous system (CNS), using a drainage catheter having first and second end portions, the method comprising:
selecting a patient suffering from or as risk of the disease;
placing the first end portion of the catheter into a cerebral ventricle of the patient; and
placing the second end portion of the catheter into a venous sinus of the patient's head to allow the patient's cerebral spinal fluid (CSF) to flow through the catheter from the ventricle to the venous sinus.

28. The method of claim 27, wherein placing the second end portion of the catheter into the venous sinus of the patient comprises placing the second end portion of the catheter into the venous sinus in a retrograde direction facing upstream to blood flow in the venous sinus.

29. The method of claim 28, further comprising creating a hole in the patient's dura through which the catheter may be inserted to place the first end portion of the catheter in the cerebral ventricle.

30. The method of claim 29, further comprising stretching the catheter such that a stretched outer diameter of the catheter is less than the diameter of the hole in the dura to facilitate insertion of the catheter the hole.

31. The method of claim 30, further comprising relaxing the catheter after insertion through the hole in the dura and allowing the outer diameter of the catheter to expand from the stretched outer diameter to a relaxed outer diameter and to sealingly engage the hole.

32. The method of claim 31, wherein placing the first end portion of the catheter and placing the second end portion of the catheter comprise placing a catheter having no flow restrictor element.

33. The method of claim 31, further comprising operably connecting a sinus piece of the catheter to a ventricular piece of the catheter,
wherein the catheter comprises two pieces, the ventricular piece comprising the first end portion and the sinus piece comprising the second end portion.

34. The method of claim 33, further comprising operably connecting a unidirectional check valve to the sinus piece and to the ventricular piece, the unidirectional check valve adapted to allow the CSF to flow from the ventricle to the sagittal sinus and to prevent flow of the patient's blood from the sagittal sinus to the ventricle.

35. The method of claim 34, wherein connecting the unidirectional check valve comprises connecting a check valve having an opening pressure of less than or equal to about 6 cm/H₂O at a flow rate of about 20 ml/hr.

36. The method of claim 35, wherein the flow of CSF from the ventricle to the venous sinus is not impeded by a flow restrictor element.

37. The method of claim 36, wherein the disease is selected from the group consisting of Guillain-Barre syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

38. The method of claim 37, wherein the venous is a sagittal sinus.

39. The method of claim 38, wherein the sagittal sinus is the superior sagittal sinus.

40. The method of claim 37, wherein the venous sinus is a transverse sinus.

41. The method of claim 36, wherein the disease is Alzheimer's type adult-onset dementia.

42. The method of claim 41, wherein the venous is a sagittal sinus.

43. The method of claim 42, wherein the sagittal sinus is the superior sagittal sinus.

44. The method of claim 41, wherein the venous sinus is a transverse sinus.

45. The method of claim 27, wherein the disease is selected from the group consisting of Guillain-Barre syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

46. The method of claim 27, wherein the disease is Alzheimer's type adult-onset dementia.

47. The method of claim 27, wherein the venous is a sagittal sinus.

48. The method of claim 47, wherein the sagittal sinus is the superior sagittal sinus.

49. The method of claim 27, wherein the venous sinus is a transverse sinus.

50. A method for treating a disease associated with increased concentration of an agent in a central nervous system (CNS), comprising
selecting a patient suffering from or as risk of the disease;
draining the patient's cerebral spinal fluid (CSF) from a cerebral ventricle of the patient to a venous sinus of the patient's head.

51. The method of claim 50, wherein the disease is selected from the group consisting of Guillain-Barré syndrome; Multiple Sclerosis (MS); Amyotrophic Lateral Sclerosis (ALS); Acquired Immune Deficiency Syndrome (AIDS); demential complex; encephalopathy, such as from rabies; encephalitis; meningitis; polio; tetanus; CNS infection; and autoimmune disease.

52. The method of claim 50, wherein the disease is Alzheimer's type adult-onset dementia.
